(19) United States
(12) Patent Application Publication
(10) Pub. No.: US 2020/0061230 A1
(43) Pub. Date: Feb. 27, 2020

(54) STERILIZATION SYSTEM AND METHOD

(71) Applicant: Fadhil Yousif Hammadi, Baghdad (IQ)

(72) Inventor: Fadhil Yousif Hammadi, Baghdad (IQ)

(21) Appl. No.: 16/113,010

(22) Filed: Aug. 27, 2018

(51) Int. Cl. A61L 9/16

(52) U.S. Cl. CPC A61L 9/16; A61L 2209/11; A61L 2209/14

(57) ABSTRACT

A sterilization system and method for the sterilization of medical and dental instruments, handpieces and burrs, tools and devices. Such a sterilization system and method compress hot air in the sterilization chamber with specific pressure and temperature values along specific interval of time to perform sterilization process. The sterilization system includes an air filter, a compressor, inlet solenoid valve, a heat exchanger, a sterilization chamber, an outlet aperture, and an outlet solenoid valve.

### BACKGROUND

All surgical instruments should be sterile before they can be safely used in the treatment of patients. Failure to use sterile instruments exposes patients to the risk of infection which may impede their recovery or, at worst, cause death. The two widely used methods of sterile medical, dental, veterinary, or other patient-care instruments are steam and dry heat sterilization methods.

Saturated steam under pressure is the most widely used and the most dependable sterilization method due to its nontoxic, inexpensive, rapidly microbicidal, sporicidal, and rapidly heats and penetrates fabrics. On the other hand, it is reported that steam method has the particular disadvantage of corroding and dulling metal and sharp edges of cutting instruments. Also, it may leave instruments wet, causing them to rust or take additional time interval during sterilization cycle for drying.

The advantages of dry heat sterilization include the following: it is nontoxic and does not harm the environment; a dry heat cabinet is easy to install and has relatively low operating costs; it penetrates materials; and it is noncorrosive for metal and sharp instruments. The disadvantages of dry heat are the slow rate of heat penetration and microbial killing makes this a time-consuming method. In addition, the high temperatures are not suitable for most materials.

Currently, the problems of dry-heat sterilization method related to long time required to complete a cycle, relatively high temperature that not suitable for most materials and the complexity, bulky and costly components of forced hot air sterilizers. These problems still the most obstacles against of extending dry-heat method widely, especially for the dental clinics that require as example, overall small size, small footprint, power efficiency, small number of parts, simplicity of design, low manufactured cost, and efficient heat distribution.

### SUMMARY

According to at least one exemplary embodiment, a sterilization system and method are described. The sterilization system and method may compress hot air in the sterilization chamber with specific pressure and temperature values along specific interval of time to perform sterilization process.

Such a system may include: a chamber which is configured to contain air for a sterilization; an inlet valve for the air inserted into the chamber; a compressor which is configured to compress the air in the chamber up to a predetermined pressure; a heater which is configured to heat the air before inserted into the chamber up to a predetermined temperature, and an outlet valve for the air released from the chamber.

In another exemplary embodiment, a sterilization method may be described. Such a method may include: opening, by a controller, an inlet valve and an outlet valve; inserting, by a compressor, air into a chamber; heating, by a heater, the air inserted into the chamber up to a predetermined temperature; closing, by the controller, the outlet valve; compressing, by the compressor, the air in the chamber up to a predetermined pressure; closing, by the controller, the inlet valve for a predetermined time; opening, by the controller, the outlet valve; releasing the pressed air from the chamber; opening, by the controller, the outlet valve and the inlet valve; and recycling, by the compressor, a fresh air through the chamber.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1 is an exemplary embodiment of a sterilization system.

FIG. 2 is an exemplary embodiment of the sterilization system under a heating interval.

FIG. 3 is an exemplary embodiment of the sterilization system under a compressing interval.

FIG. 4 is an exemplary embodiment of the sterilization system under a sterilizing interval.

FIG. 5 is an exemplary embodiment of the sterilization system under a pressure release interval.

FIG. 6 is an exemplary embodiment of the sterilization system under a cooling interval.

FIG. 7 is an exemplary configuration of monitor and control temperature sensors for the sterilization system.

FIG. 8 is an exemplary heater of a chamber body for the sterilization system.

FIG. 9 is another exemplary heater for the sterilization system.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

According to an exemplary embodiment, and referring generally to the Figures, various exemplary implementations of a system and a method for the sterilization of medical and dental instruments, handpieces and burrs, tools and devices may be disclosed. Such a sterilization system and method may compress hot air in the sterilization chamber with specific pressure and temperature values along specific interval of time to perform sterilization process. Accordingly, the sterilization system and method may be: low expensive, safe and fast hot-air sterilization system; durable and requiring little maintenance; less harmful to instruments than other devices; being used with no steam or chemicals; and small enough to be provided at multiple convenient locations such as each patient room or operating room. According to an exemplary embodiment, the sterilization system and method may provide a sterilizer with rapid heat distribution suitable for sterilizing dental implements and handpieces that cannot withstand the effects of high temperature and prolonged thermal exposure. Also, in an exemplary embodiment, the sterilization system and method may provide rapid and convenient sterilization of needed implements so as to avoid bagging or wrapping dental implements to be sterilized.

According to an exemplary embodiment, a sterilization system may include a housing combine a sterilization chamber and a base which may define an air compressor and any kind of heaters or a heat exchanger which may has an electric heater and air ducts joint by pipe to the chamber. Also, in an exemplary embodiment, implements to be sterilized may be supported in a caddy suspended within the chamber. The chamber may be sealed off to keep air pressure so that cannot leak to outside. Fresh air may be compressed into the air heater or heat exchanger, heated, and forced into the sterilization chamber. Hot air may compress in the chamber under adequate pressure and temperature values to sterilizing the implements efficiently and completely under less temperature degree and time interval than conventional hot-air sterilizers.

Further, according to an exemplary embodiment, the sterilization system may include a control unit for receiving input data from a user, and the control unit may include a timer and a thermostat control. The user may input data to the control unit corresponding to a desired heating interval and a desired cooling interval. The user may also input to the control unit a desired chamber pressure and temperature during the heating interval and/or during the cooling interval. According to an exemplary embodiment, a thermostat control and timer of the control unit may cooperate to control the heater and compressor to regulate the chamber air temperature and pressure at the desired set point.

Conventional types of dry heat sterilizer, generally, utilize static or forced hot air circulating techniques with zero pressure value (relative pressure value P=0 bar) as Gauge pressure is the pressure relative to the ambient pressure. On the other hand, according to an exemplary embodiment, the sterilization system and method may utilize hot air with pressure more than 0 bar. This mechanism may depend on compress fresh air inside the chamber by external air compressor through external heater or heat exchanger to elevate pressure and temperature inside the chamber to the selected values (for example, relative pressure value P=1, 2, 3 bar or more). According to an exemplary embodiment, there may be three parameters of steam sterilization: pressure; temperature; and time, while there are generally two parameters for conventional dry heat sterilizations: temperature; and time. Therefore, in an exemplary embodiment, the sterilization system and method may provide additional parameter for dry heat sterilization process: pressure.

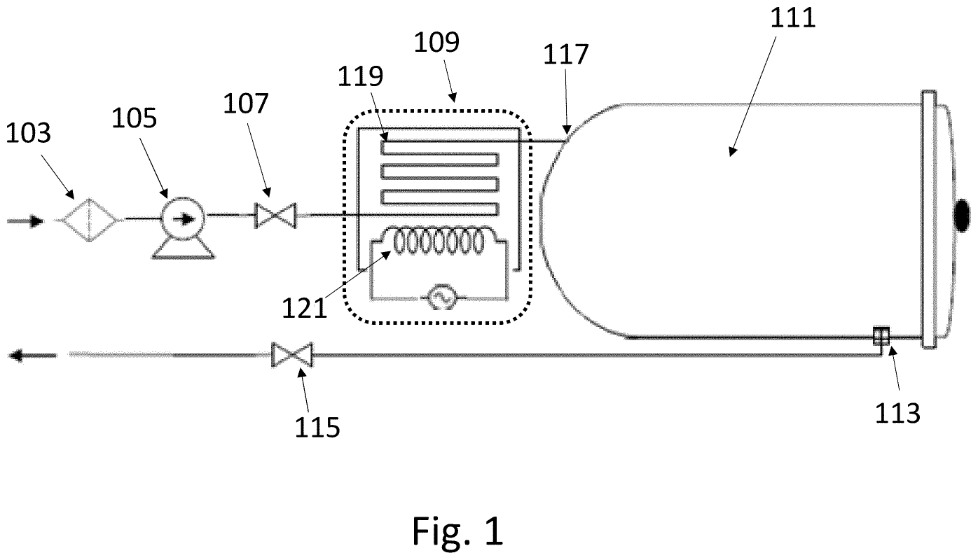

Turning now to exemplary FIG. 1, FIG. 1 displays an exemplary embodiment of a sterilization system. According to an exemplary embodiment, a sterilization system and method may include an air filter 103, a compressor 105, inlet valve 107, a heater 109, a sterilization chamber 111, an outlet aperture 113, and an outlet valve 115. In an exemplary embodiment, the air filter 103 may deliver clean air from ambient to the compressor 105, and the compressor 105 may compress air and deliver it to the heater 109. Here, the inlet valve 107 may allow air to pass when the compressor 105 running and may close when the compressor 105 stops. According to an exemplary embodiment, any kind of heaters may be used as desired. In an exemplary embodiment, to heat the compressed air to specific temperature and delivered it to the sterilization chamber 111, the heater 109 may be a kind of heat exchanger which may include air ducts 119 inside and heat the compressed air in the air ducts 119, for example, with an electrical heater 121. The sterilization chamber 111 may have an inlet aperture 117 to enter hot air and the outlet aperture 113 to drain cold air or used air, and the outlet aperture 113 may have a filter to protect the outlet valve 115 from solid impurities. The outlet valve 115 may open, in the beginning of cycle to drain cold air to outside, and open in the end of cycle to release pressure of used air to outside and finally open for refreshing the chamber in cooling interval.

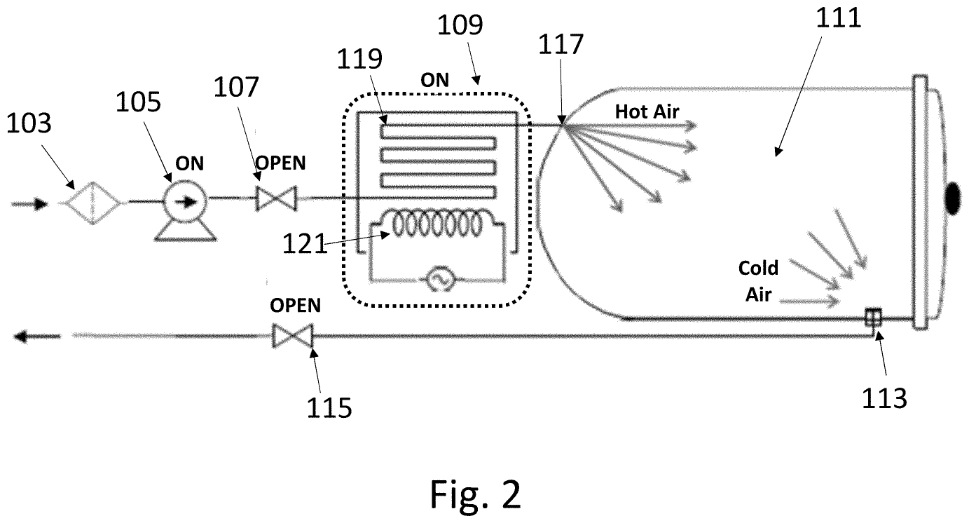

Turning now to exemplary FIG. 2, FIG. 2 displays an exemplary embodiment of the sterilization system under a heating interval. According to an exemplary embodiment, in the heating interval, the compressor 105 may receive ambient air through the air filter 103 to deliver it through the inlet valve 107 to the heater 109. The inlet valve 107 may allow air to pass from compressor 105 to the heater 109 and may not allow go back for reverse direction. This inlet valve 107 may open only when the compressor 105 run and the inlet valve 107 may close when the compressor 105 stops. As mentioned above, in a case where the heat exchanger is used as the heater 109, the heat exchanger 109 may has air ducts 119 that may heat the compressed air by, for example, an electric heater 121 to a desired temperature degree and may deliver it to the chamber 111 through the inlet aperture 117. In the chamber 111, the compressed hot air may enter from the inlet aperture 117 into the chamber 111 or through turbulence facilitates to distribute heated air uniformly within the chamber 111. In an exemplary embodiment, hot air may push cold air down to drain it through the outlet aperture 113, and then to the ambient through the outlet valve 115 in the beginning of sterilization cycle. These procedures may be performed as forced convection method that produces more uniform temperature inside chamber space during short time. The outlet valve 115 may open two times: in the beginning of sterilization cycle (heating interval); and in the end of this cycle (pressure release interval and cooling interval).

Referring still to exemplary FIG. 2, the outlet valve 115 may open to allow for cold air to pass outside and may stay open for an interval of time, depend on compressor 105 flow rate, or by measure air temperature in the outlet aperture 113 to close the outlet valve 115 when the temperature increase to high level, which indicate that cold air completely go out.

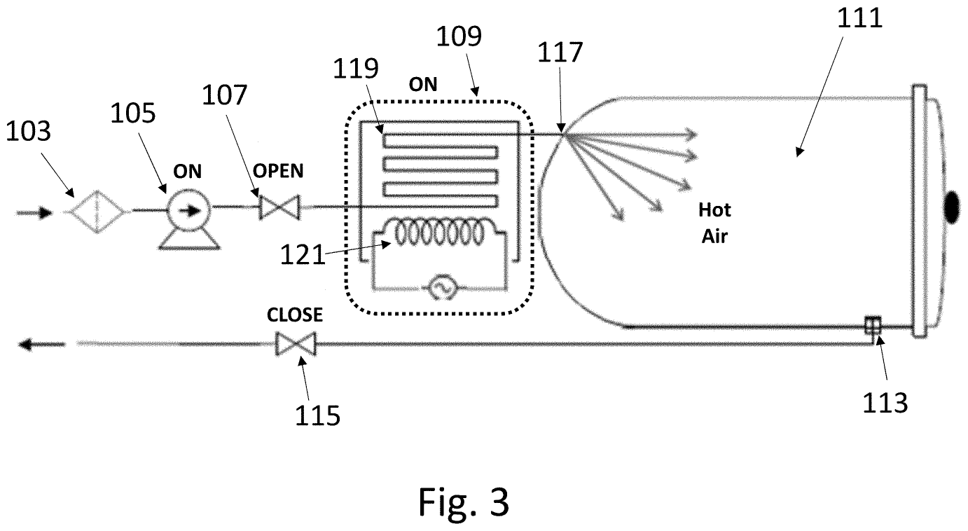

Turning now to exemplary FIG. 3, FIG. 3 displays an exemplary embodiment of the sterilization system under a compressing interval. According to an exemplary embodiment, in the compressing interval, the outlet valve 115 may close while the compressor 105 continue running to elevate pressure of hot air inside the chamber 111 to desired pressure and temperature values.

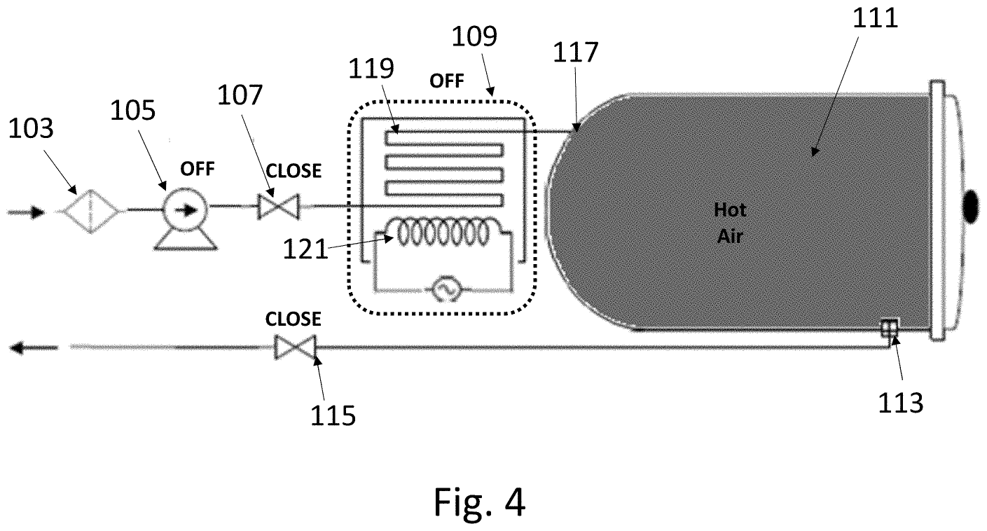

Turning now to exemplary FIG. 4, FIG. 4 displays an exemplary embodiment of the sterilization system under a sterilizing interval. According to an exemplary embodiment, in the sterilizing interval, the compressor 105 may stop, the inlet valve 107 may close, the heater 109 may off and the outlet valve 115 may close to keep steady pressure and temperature along the sterilizing time.

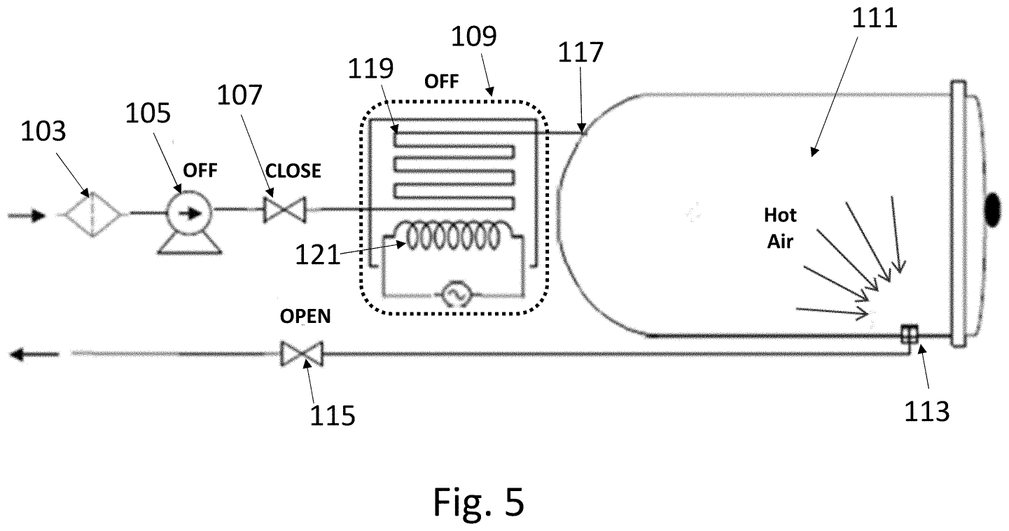

Turning now to exemplary FIG. 5, FIG. 5 displays an exemplary embodiment of the sterilization system under a pressure release interval. According to an exemplary embodiment, at the finish of the sterilizing interval, the pressure release interval may start, the outlet valve 115 may open to release pressure of hot air and may stay open for starting of a cooling interval.

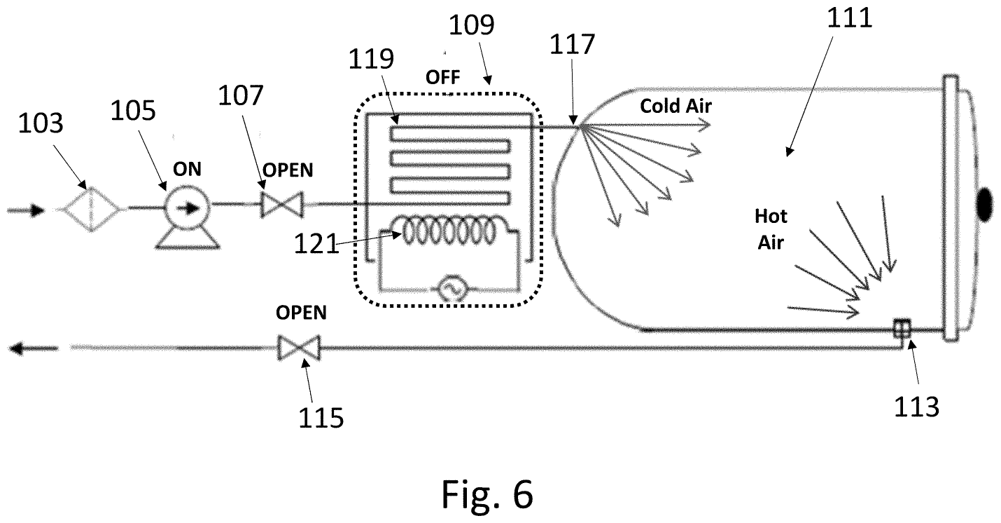

Turning now to exemplary FIG. 6, FIG. 6 displays an exemplary embodiment of the sterilization system under a cooling interval. According to an exemplary embodiment, during the cooling interval, the compressor 105 may run, the inlet valve 107 may open, the heater 109 may off and the outlet valve 115 may stay open to circulate fresh air inside the chamber during the cooling interval.

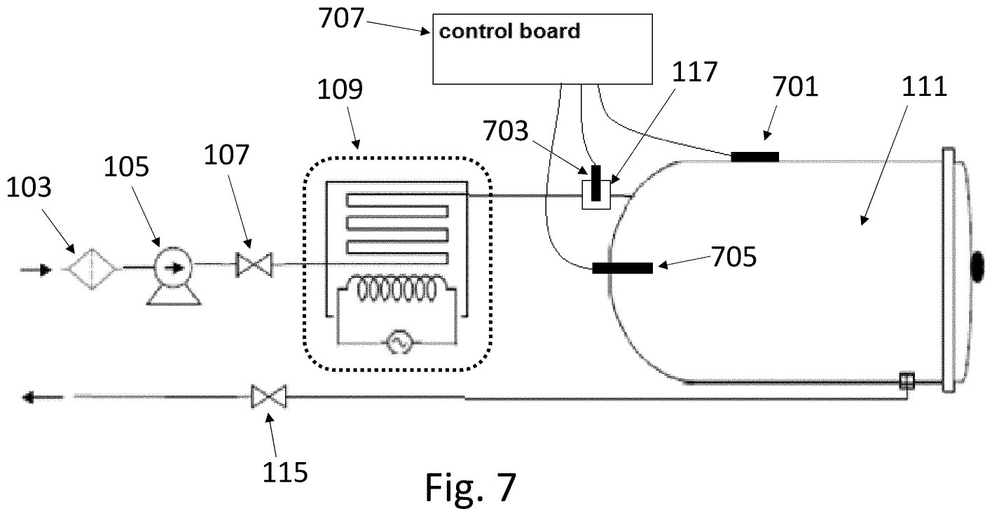

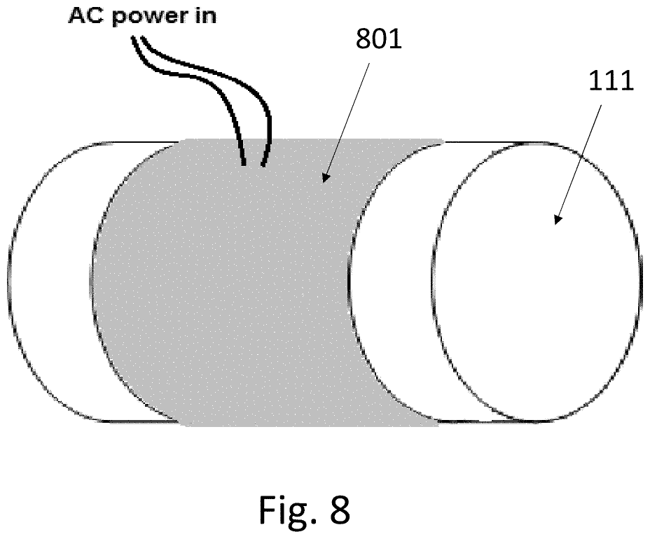

Turning now to exemplary FIG. 7, FIG. 7 displays an exemplary configuration of monitor and control temperature sensors for the sterilization system. According to an exemplary embodiment, to monitor and control the temperature of the sterilization system, three thermo sensors may be used for: the chamber wall temperature 701; the input hot air temperature 703; and the temperature of sterilization volume inside the chamber 705. In an exemplary embodiment, the controller 707 may gather temperature information from the sensors (701, 703 and 705) to control the inlet valve 107, the compressor 105, the heater 109 and/or the outlet valve 115 so that the predetermined pressure in the chamber 111 may be controlled and maintained with the predetermined temperature for a predetermined time. For example, the third sensor 705 may be a sensor that is used to monitor and control the predetermined temperature of sterilization process inside sterilization volume. In an exemplary embodiment, a specific temperature may also be achieved by using the second sensor 703 in the inlet aperture 117 of sterilization chamber 111, which is to adjust the temperature of hot air by controlling the heater 109 to achieve a desired temperature. Further, there may be some flexibility in the setting of the desired temperature, for example, if the sterilize temperature of 134° is required to destroy microorganisms, then more degrees of: 135°, 136°, 137°, or etc. may perform better actions in the disinfection. Also, the first sensor 701 may be used to control the temperature of the chamber body that may be heated as shown in exemplary FIG. 8.

Turning now to exemplary FIG. 8, FIG. 8 displays an exemplary heater of a chamber body for the sterilization system. According to an exemplary embodiment, the chamber 111 may be heated for the predetermined temperature. In an exemplary embodiment, a circular heater 801 which may wrap around the chamber 111 may be used to heat, for example, as an electric heater, the chamber body for keeping the temperature of the sterilization volume inside the chamber in homogenous distribution. According to an exemplary embodiment, the circular heater 801 may be used to preheat the chamber 111, and without being limited to the electric heater, the heater 801 may use any other means of heating as desired.

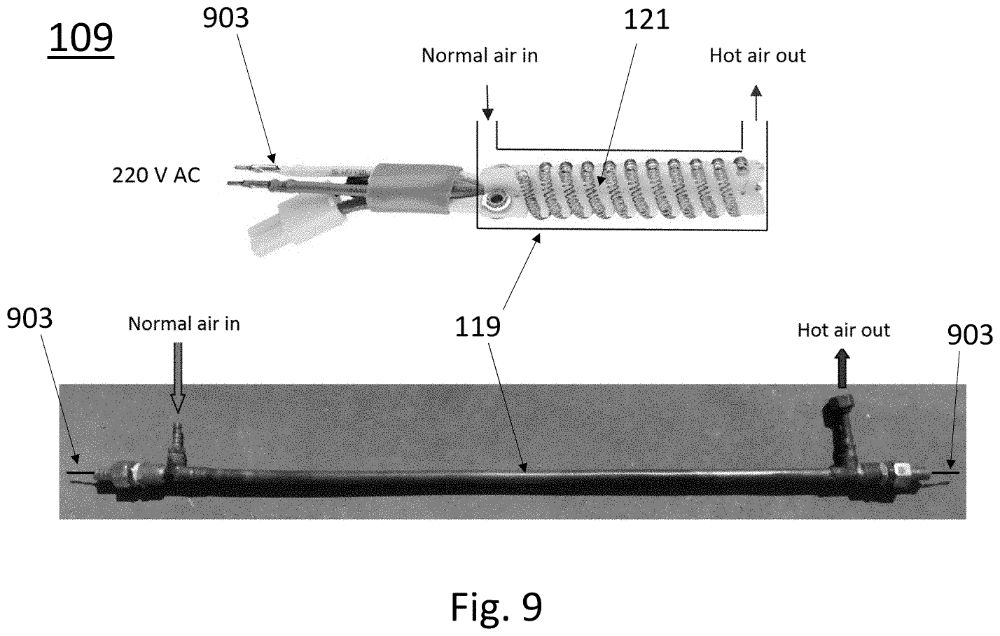

Turning now to exemplary FIG. 9, FIG. 9 displays another exemplary heater for the sterilization system. According to an exemplary embodiment, as mentioned above, the heater 109 may be a heat exchanger and apply any electric coil heater 121 in order to heat normal air that injected through input opening to produce hot air through output opening. According to an exemplary embodiment, there may be a coil heater 901 inside the air duct 119, the air duct may be made with a copper or any material as desired, and the air duct 119 may also be insulated by heat resistant material shield such as glass. Electric poles 903 of the heater coil 121 may be on each terminal of the air duct, but the configurations may be varied as desired. Normal air, that injected through the input opening, flow along the duct 119 in touch directly with the hot electric heater coil 121 to go out as hot air through the output opening. The desired temperature degree may be adjusted by controlling the active current of the electric heater.

According to an exemplary embodiment, any kind of valve may be used for the inlet valve 107 and the outlet valve 115 as desired. For example, to control the valves depending on a specific interval, a solenoid valve may be used. Also, in an exemplary embodiment, a specific pressure may be achieved by controlling the operation time of the compressor 105, for example, 0.5, 1, 1.5 minutes of the compressor operation time may give 1, 2, 3 bar of air pressure in the sterilization chamber 111 depending on compressor sizes. Further, same flexibility mentioned above regarding the temperature may also exist with respect to the pressure. According to an exemplary embodiment, unlike conventional steam sterilizers, the temperature and pressure may be independent parameters so that the temperature and pressure may be adjusted separately by controlling the heater 109 and the operation time of the compressor 105 respectively.

According to an exemplary embodiment, as described above, the sterilization system and method may utilize “pressure” as a new parameter in dry heat sterilizers in order to decrease temperature degree and time interval that required to perform efficiently microbicidal. Relatively low temperatures required for decontamination of materials like plastic (include bagging or wrapping medical instruments), fabrics or even food products. Therefore, utilizing of pressure parameter may open new aspirations for decontamination by dry air method, more pressure less temperature and less time required for efficient decontamination.

According to an exemplary embodiment, the sterilization system and method may provide sterilization with short cycle time so as to reduce the required inventory of dental implements, i.e. the implements can be sterilized between patients. Also, the sterilization system and method may provide a sterilizer of convenient size and footprint to better utilize limited counter-top space within a patient room. Further, the sterilizer may be of a size and shape to facilitate table-top or counter-top use. Patient confidence may be enhanced by permitting visual inspection of sterilization procedures.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art (for example, features associated with certain configurations of the invention may instead be associated with any other configurations of the invention, as desired).

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A sterilization system comprising:
a chamber configured to contain air for a sterilization;
an inlet valve for the air inserted into the chamber;
a compressor configured to compress the air in the chamber up to a predetermined pressure;
a heater configured to heat the air up to a predetermined temperature, and an outlet valve for the air released from the chamber.

2. The system of claim 1, wherein the chamber includes an inlet aperture and an outlet aperture.

3. The system of claim 2, wherein the air inserted into the chamber via the inlet aperture and released via outlet aperture.

4. The system of claim 2, wherein the air inserted into the chamber as air turbulence by the inlet aperture to distribute the air uniformly within the chamber.

5. The system of claim 1 further comprises at least one air filter to insert clean air into the chamber or to protect the outlet valve from impurities.

6. The system of claim 1, wherein the inlet valve is an inlet solenoid valve, and the outlet valve is an outlet solenoid valve.

7. The system of claim 1 further comprises a controller, wherein the controller is configured to control at least one of the inlet valve, the compressor, the heater and the outlet valve so that the predetermined pressure in the chamber is maintained with the predetermined temperature for a predetermined time.

8. The system of claim 1, wherein the inlet valve, the compressor, the heater, the chamber and the outlet valve are connected via an air duct.

9. The system of claim 1, wherein the heater is a heat exchanger.

10. The system of claim 9, wherein the heat exchanger is configured to heat the air in an air duct with an electrical heater.

11. A method of sterilization comprising:
opening, by a controller, an inlet valve and an outlet valve;
inserting, by a compressor, air into a chamber;
heating, by a heater, the air up to a predetermined temperature;
closing, by the controller, the outlet valve;
compressing, by the compressor, the air in the chamber up to a predetermined pressure;
closing, by the controller, the inlet valve for a predetermined time;
opening, by the controller, the outlet valve;
releasing the pressed air from the chamber;
opening, by the controller, the outlet valve and the inlet valve; and
recycling, by the compressor, a fresh air through the chamber.

12. The method of claim 11, wherein the chamber includes an inlet aperture and an outlet aperture.

13. The method of claim 12, wherein the air inserted into the chamber via the inlet aperture and released via outlet aperture.

14. The method of claim 12, wherein the air inserted into the chamber as air turbulence by the inlet aperture to distribute the air uniformly within the chamber.

15. The method of claim 11, wherein the air is inserted or released via at least one air filter so that a clean air is inserted into the chamber or released to the outlet valve.

16. The method of claim 11, wherein the inlet valve is an inlet solenoid valve, and the outlet valve is an outlet solenoid valve.

17. The method of claim 11, wherein the controller controls at least one of the inlet valve, the compressor, the heater and the outlet valve so that the predetermined pressure in the chamber is maintained with the predetermined temperature for the predetermined time.

18. The method of claim 11, wherein the inlet valve, the compressor, the heater, the chamber and the outlet valve are connected via an air duct.

19. The method of claim 11, wherein the heater is a heat exchanger.

20. The method of claim 19, wherein the heat exchanger is configured to heat the air in an air duct with an electrical heater.
